(19) United States
(12) Patent Application Publication
(10) Pub. No.: US 2014/0140983 A1
(43) Pub. Date: May 22, 2014

(54) METHOD FOR TREATMENT OF AUTONOMIC NERVOUS SYSTEM DISORDERS

(71) Applicant: Andrew M. Blumenfeld, Del Mar, CA (US)

(72) Inventor: Andrew M. Blumenfeld, Del Mar, CA (US)

(73) Assignee: ALLERGAN, INC., Irvine, CA (US)

(21) Appl. No.: 14/130,854

(22) PCT Filed: Jul. 6, 2012
(86) PCT No.: PCT/US12/45766

§ 371 Date: Jan. 22, 2014

Related U.S. Application Data
(60) Provisional application No. 61/505,960, filed on Jul. 8, 2011.

(51) Int. Cl. A61K 38/48

(52) U.S. Cl. CPC A61K 38/4893
USPC 424/94.67

(57) ABSTRACT

Botulinum toxin, among other presynaptic neurotoxins is used for the treatment of autonomic nervous system disorders such as irritable bowel syndrome. The neurotoxin is delivered to target the sympathetic ganglion. Exemplary delivery is carried out by way of injection.

### CROSS REFERENCE

This application claims the benefit of U.S. Provisional Patent Application Ser. No. 61/505,960, filed on Jul. 8, 2011, the entire disclosure of which is incorporated herein by this specific reference.

### FIELD OF THE INVENTION

The present invention relates to methods for treating autonomic nervous system disorders such as irritable bowel syndrome and other conditions.

### BACKGROUND

The sympathetic nervous system operates through a series of interconnected neurons. Sympathetic nerves originate in the spinal cord in the intermediolateral cell column, extending from the first thoracic segment to the third lumbar segment. Axons of these nerves leave the spinal cord in the ventral rami of the spinal nerves, and then separate out as white rami which connect to peripheral ganglia. These ganlia form the sympathetic trunks which lie on either side of the spinal cord.

Spinal cord sympathetic neurons are termed presynaptic (or preganglionic) neurons, while peripheral sympathetic neurons are termed postsynaptic (or postganglionic) neurons.

Preganglionic sympathetic neurons release acetylcholine, which binds and activates nicotinic acetylcholine receptors on postganglionic neurons. In response to this stimulus, postganglionic neurons principally release norepinephrine. Prolonged activation can elicit the release of adrenaline from the adrenal medulla.

Once released, norepinephrine binds to adrenergic receptors on peripheral tissues. Binding to adrenergic receptors causes the effects seen during the fight-or-flight response. These include pupil dilation, increased heart rate, occasional vomiting, and increased blood pressure. Increased sweating is also seen due to binding of cholinergic receptors of the sweat glands.

The ganglia include not just the sympathetic trunks but also the cervical ganglia (superior, middle and inferior), which sends sympathetic fibers to the head and thorax organs, and the celiac and mesenteric ganglia (which send sympathetic fibers to the gut).

The sympathetic nervous system can accelerate heart rate; widen bronchial passages; decrease motility of the large intestine; constrict blood vessels; increase peristalsis in the esophagus; cause pupil dilation, piloerection and perspiration and raise blood pressure. Afferent messages carry sensations such as pain.

Disorders of the sympathetic nervous system can result in gastrointestinal disorders such as irritable bowel syndrome. Irritable bowel syndrome is a functional gastrointestinal disorder characterized by, for example, chronic disturbance of bowel habit (i.e. constipation or diarrhea) in association with abdominal pain, discomfort and/or bloating without any detectable organic disease in the gastrointestinal tract. There may also be urgency for bowel movements, a feeling of incomplete evacuation, bloating or abdominal distention. According to the diagnostic criteria for irritable bowel syndrome (Rome III Criteria) published in 2006, irritable bowel syndrome is categorized as irritable bowel syndrome with constipation, irritable bowel syndrome with diarrhea, mixed irritable bowel syndrome or unclassified irritable bowel syndrome.

Irritable bowel syndrome greatly worsens the quality of life and the work productivity of the patient, and represents a therapeutic challenge to both clinicians and developers of pharmaceuticals. The uncertainty and variety of causes, as well as the variable nature of symptomatic expression greatly complicates the task of treating this disorder.

Treatments that attempt to relieve symptoms of conditions such as irritable bowel syndrome include dietary adjustments, medication and psychological interventions. Known treatments of conditions such as irritable bowel syndrome include the use of antispastic drugs such as anticholinergic drug, antidiarrheal drugs such as opioid receptor agonist, or bulking-agents or probiotics for regulating the enteric environment.

Further known treatments of conditions such as irritable bowel syndrome include administration of a botulinum toxin to the bowel wall at multiple sites. Other known treatments of conditions such as irritable bowel syndrome include administering a botulinum toxin to the head, neck and/or shoulder location of a patient (US Patent Application Publication Number 2007/0048334).

Botulinum toxin type A is the most lethal natural biological agent known to man. About 50 picograms of botulinum toxin type A (available from Allergan, Inc., of Irvine, Calif. under the tradename BOTOX®) is an LD₅₀ in mice. One unit (U) of botulinum toxin is defined as the LD₅₀ upon intraperitoneal injection into female Swiss Webster mice weighing 18-20 grams each. Seven immunologically distinct botulinum neurotoxins have been characterized, these being respectively botulinum neurotoxin serotypes A, B, C₁, D, E, F and G, each of which is distinguished by neutralization with type-specific antibodies. The different serotypes of botulinum toxin vary in the animal species that they affect and in the severity and duration of the paralysis they evoke. The botulinum toxins apparently bind with high affinity to cholinergic motor neurons, are translocated into the neuron and block the release of acetylcholine.

Botulinum toxins have been used in clinical settings for the treatment of neuromuscular disorders characterized by hyperactive skeletal muscles. Botulinum toxin type A has been approved by the U.S. Food and Drug Administration for the treatment of blepharospasm, strabismus, hemifacial spasm, cervical dystonia, and migraine headaches. Botulinum toxin type B has also been approved by the FDA for the treatment of cervical dystonia. Clinical effects of peripheral intramuscular Botulinum toxin type A are usually seen within one week of injection. The typical duration of symptomatic relief from a single intramuscular injection of Botulinum toxin type A averages about three months.

It has been reported that Botulinum toxin type A has been used in clinical settings as follows:

about 75-125 U (U) of BOTOX® per intramuscular injection (multiple muscles) to treat cervical dystonia;

5-10 U of BOTOX® per intramuscular injection to treat glabellar lines (brow furrows) (5 U injected intramuscularly into the procerus muscle and 10 U injected intramuscularly into each corrugator supercilii muscle);

about 30-80 U of BOTOX® to treat constipation by intrasphincter injection of the puborectalis muscle;

about 1-5 U per muscle of intramuscularly injected BOTOX® to treat blepharospasm by injecting the lateral pre-tarsal orbicularis oculi muscle of the upper lid and the lateral pre-tarsal orbicularis oculi of the lower lid;

to treat strabismus, extraocular muscles have been injected intramuscularly with between about 1-5 U of BOTOX®, the amount injected varying based upon both the size of the muscle to be injected and the extent of muscle paralysis desired (i.e. the amount of diopter correction desired);

to treat upper limb spasticity following stroke by intramuscular injections of BOTOX® into five different upper limb flexor muscles, as follows:

(a) flexor digitorum profundus: 7.5 U to 30 U

(b) flexor digitorum sublimus: 7.5 U to 30 U

(c) flexor carpi ulnaris: 10 U to 40 U

(d) flexor carpi radialis: 15 U to 60 U

(e) biceps brachii: 50 U to 200 U.

Each of the five indicated muscles has been injected at the same treatment session, so that the patient receives from 90 U to 360 U of upper limb flexor muscle BOTOX® by intramuscular injection at each treatment session.

To treat migraine, pericranial (symmetrically into glabellar, frontalis and temporalis muscles) injection of 25 U of BOTOX® has showed significant benefit as a prophylactic treatment compared to vehicle as measured by decreased measures of migraine frequency, maximal severity, associated vomiting and acute medication use over the three month period following the 25 U injection.

Additionally, intramuscular Botulinum toxin has been used in the treatment of tremor in patients with Parkinson's disease, although it has been reported that results have not been impressive. Marjama-Jyons, J., et al., Tremor-Predominant Parkinson's Disease, Drugs & Aging 16(4); 273-278:2000.

In addition to having pharmacologic actions at the peripheral location, botulinum toxins may also have inhibitory effects in the central nervous system. Work by Weigand et al., Naunyn-Schmiedeberg's Arch. Pharmacol. 1976; 292, 161-165, and Habermann, Naunyn-Schmiedeberg's Arch. Pharmacol. 1974; 281, 47-56 showed that Botulinum toxin is able to ascend to the spinal area by retrograde transport. As such, a Botulinum toxin injected at a peripheral location, for example intramuscularly, may be retrograde transported to the spinal cord.

U.S. Pat. No. 5,989,545 discloses that a modified Clostridial neurotoxin or fragment thereof, preferably a Botulinum toxin, chemically conjugated or recombinantly fused to a particular targeting moiety can be used to treat pain by administration of the agent to the spinal cord.

A Botulinum toxin has also been proposed for the treatment of rhinorrhea, hyperhydrosis and other disorders mediated by the autonomic nervous system (U.S. Pat. No. 5,766,605), tension headache, (U.S. Pat. No. 6,458,365), migraine headache (U.S. Pat. No. 5,714,468), post-operative pain and visceral pain (U.S. Pat. No. 6,464,986), pain treatment by intraspinal toxin administration (U.S. Pat. No. 6,113,915), Parkinson's disease and other diseases with a motor disorder component, by intracranial toxin administration (U.S. Pat. No. 6,306,403), hair growth and hair retention (U.S. Pat. No. 6,299,893), psoriasis and dermatitis (U.S. Pat. No. 5,670,484), injured muscles (U.S. Pat. No. 6,423,319, various cancers (U.S. Pat. No. 6,139,845), pancreatic disorders (U.S. Pat. No. 6,143,306), smooth muscle disorders (U.S. Pat. No. 5,437,291, including injection of a botulinum toxin into the upper and lower esophageal, pyloric and anal sphincters)), prostate disorders (U.S. Pat. No. 6,365,164), inflammation, arthritis and gout (U.S. Pat. No. 6,063,768), juvenile cerebral palsy (U.S. Pat. No. 6,395,277), inner ear disorders (U.S. Pat. No. 6,265,379), thyroid disorders (U.S. Pat. No. 6,358,513), parathyroid disorders (U.S. Pat. No. 6,328,977). Additionally, controlled release toxin implants are known (see e.g. U.S. Pat. Nos. 6,306,423 and 6,312,708).

In addition, a clostridial toxin such as a botulinum toxin can be modified such that the toxin has an altered cell targeting capability for a neuronal or non-nuronal cell of interest. Called re-targeted endopeptidases or Targeted Vesicular Exocytosis Modulator Proteins (TVEMPs), these molecules achieve their exocytosis inhibitory effects by targeting a receptor present on the neuronal or non-neuronal target cell of interest. This re-targeted capability is achieved by replacing the naturally-occurring binding domain of a clostridial toxin with a targeting domain showing a selective binding activity for a non-clostridial toxin receptor present in a cell of interest. Such modifications to the binding domain result in a molecule that is able to selectively bind to a non-clostridial toxin receptor present on the target cell. A re-targeted endopeptidase can bind to a target receptor, translocate into the cytoplasm, and exert its proteolytic effect on the SNARE complex of the neuronal or non-neuronal target cell of interest.

The sympathetic ganglia can be treated with Botulinum toxin. This has not been done clinically to date. The treatment would result in blockade of the pre-synaptic acetylcholine activation of the ganglion.

### DESCRIPTION

Certain embodiments of the invention utilize a method of administering compositions comprising Botulinum toxins and like substances to treat autonomic nervous system disorders such as, for example, irritable bowel syndrome, and the like. In certain preferred embodiments, the method of administration can be, for example, injection of Botulinum toxin to specific sympathetic ganglia and/or to the region thereof.

The following definitions apply herein:

The singular forms “a”, “an” and “the” include plural references unless the context dictates otherwise.

“About” means approximately or nearly and in the context of a numerical value or range set forth herein means .+−.10% of the numerical value or range recited or claimed.

“Administration” or “administering” refers to percutaneous administration to or to the vicinity of one or more of the ganglia in the sympathetic chain, which can be performed under fluoroscopy, direct vision, direct surgical vision, endoscopic delivery, and the like. These sites include, for example, the superior mesenteric ganglion.

“Affliction” includes a disease, disorder, problem and/or a cosmetically undesirable state or condition in an individual.

“Alleviating” means a reduction in the occurrence of a symptom related to an autonomic nervous system disorder such as irritable bowel syndrome. For example, alleviating can include some reduction, significant reduction, near total reduction, and total reduction of a symptom related to irritable bowel syndrome. An alleviating effect may not appear clinically for a number of days (for example from between 1 to 7 days) after administration of a Botulinum toxin to a patient.

“Botulinum toxin” means a Botulinum neurotoxin as either pure toxin (i.e. about 150 kDa weight molecule) or as a complex (i.e. about 300 to about 900 kDa weight complex comprising a neurotoxin molecule and one or more associated non-toxic molecules), and excludes botulinum toxins which are not neurotoxins such as the cytotoxic botulinum toxins C2 and C3, but includes recombinantly made, hybrid, modified, and chimeric botulinum toxins.

“Endopeptidase” means a biologically active molecule with a specific affinity for a cell surface receptor of sensory neurons (also known as afferent or receptor neurons). Sensory neurons carry nerve impulses from receptors or sense organs towards the central nervous system. Endopeptidases such as TVEMPs decrease the effects of sensory afferents, including conditions that are predominantly motor in origin. See, for example, U.S. Pat. No. 7,658,933 to Foster et al., titled “Non-Cytoxtoxic Protein Conojugates”; U.S. Pat. No. 7,659,092 to Foster et al., titled “Fusion Proteins”; and U.S. Ser. No. 12/303,078 to Foster et al., titled “Treatment of Pain”.

“Improved patient function” means an improvement measured by factors such as reduced abdominal pain, reduced disturbance of bowel habit (e.g. reduced constipation or reduced diarrhea), reduced bloating, reduced urgency for bowel movements, reduced feeling of incomplete evacuation, healthier attitude, and more varied lifestyle. Improved patient function is synonymous with an improved quality of life (QOL). QOL can be assessed using, for example, the known SF-12 or SF-36 health survey scoring procedures. SF-36 assesses a patient's physical and mental health in the eight domains of physical functioning, role limitations due to physical problems, social functioning, bodily pain, general mental health, role limitations due to emotional problems, vitality, and general health perceptions. Scores obtained can be compared to published values available for various general and patient populations.

“Region” or “vicinity” means in the surrounding or nearby area.

“Treating” means to alleviate (or to eliminate) at least one symptom related to an autonomic nervous system disorder (for example, irritable bowel syndrome), either temporarily or permanently.

Unless defined otherwise, all technical and scientific terms used herein have the same meaning as commonly understood to one of ordinary skill in the art to which the present invention belongs. Although any methods, devices, and materials similar or equivalent to those described herein can be used in the practice or testing of the invention, the preferred methods, devices, and materials are described herein.

Presently preferred embodiments of the invention include administration of a Botulinum toxin to specific sympathetic ganglia and/or to the region thereof. Injections at a sympathetic ganglion level can allow the Botulinum toxin to gain access to the preganglionic acetylcholine secreting nerve terminals. In certain embodiments, the administration of a Botulinum toxin to specific sympathetic ganglia and/or to the region thereof, leads to the decrease of acetylcholine release at this local level for at least 1 month; in certain other embodiments for at least 2 months; and in still certain other embodiments for at least 3 months.

In certain embodiments of the invention, a Botulinum toxin is directly administration to the mesenteric ganglia and/or to the region thereof. In certain preferred embodiments, a Botulinum toxin is directly administered to the superior mesenteric ganglia and/or to the region thereof. The superior mesenteric ganglion is the synapsing point for one of the pre- and post-synaptic nerves of the sympathetic division of the autonomous nervous system. This nerve goes on to innervate the small intestine, the ascending colon and the transverse colon of the large intestine. In certain other embodiments, a Botulinum toxin is directly administered to the inferior mesenteric ganglia and/or to the region thereof.

The botulinum toxin can be selected from the group consisting of botulinum toxin types A, B, C, D, E, F and G. Botulinum toxin type A is a preferred botulinum toxin.

Botulinum toxins for use according to certain embodiments of the present invention can be stored in lyophilized, vacuum dried form in containers under vacuum pressure or as stable liquids. Prior to lyophilization the Botulinum toxin can be combined with pharmaceutically acceptable excipients, stabilizers and/or carriers, such as albumin. The lyophilized material can be reconstituted with saline or water to create a solution or composition containing the botulinum toxin to be administered to the patient.

Exemplary, commercially available, Botulinum toxin containing compositions include, but are not limited to, BOTOX® (Botulinum toxin type A neurotoxin complex with human serum albumin and sodium chloride) available from Allergan, Inc., of Irvine, Calif. in 100 unit vials as a lyophilized powder to be reconstituted with 0.9% sodium chloride before use), DYSPORT® (Clostridium Botulinum type A toxin haemagglutinin complex with human serum albumin and lactose in the formulation, available from Ipsen Limited, Berkshire, U.K. as a powder to be reconstituted with 0.9% sodium chloride before use) which can be used at about 3 to about 4 times the amounts of BOTOX® as set forth herein in each instance, and MYOBLOC® (an injectable solution comprising Botulinum toxin type B, human serum albumin, sodium succinate, and sodium chloride at about pH 5.6, available from Solstice Neurosciences, Inc., South San Francisco, Calif.) which can be used at about 30 to about 50 times the amounts of BOTOX® as set forth herein in each instance, as known in the art. XEOMIN® (a 150 kDa Botulinum toxin type A formulation available from Merz Pharmaceuticals, Potsdam, Germany) is another useful neurotoxin which can be used at about 1 to about 2 times the amounts of BOTOX® as set forth herein in each instance.

In additional embodiments, no less than about 10 U and no more about 400 U of BOTOX®; no less than about 30 U and no more than about 1600 U of DYSPORT®, and; no less than about 250 U and no more than about 20000 U of MYOBLOC® are administered per site, per patient treatment session.

In still further embodiments, no less than about 20 U and no more about 300 U of BOTOX®; no less than about 60 U and no more than about 1200 U of DYSPORT®, and; no less than about 1000 U and no more than about 15000 U of MYOBLOC® are administered per site, per patient treatment session.

In certain embodiments the composition only contains a single type of Botulinum toxin, such as, for example, type A, as the active ingredient to suppress neurotransmission. Nevertheless, in other embodiments other therapeutic compositions may include two or more types of Botulinum toxins. For example, a composition administered to a patient may include Botulinum toxin type A and Botulinum toxin type B. Administering a single composition containing two different neurotoxins can permit the effective concentration of each of the neurotoxins to be lower than if a single neurotoxin is administered to the patient while still achieving the desired therapeutic effects. The composition administered to the patient may also contain other pharmaceutically active ingredients, such as, for example, protein receptor or ion channel modulators, or the like, in combination with the neurotoxin or neurotoxins. These modulators can contribute to the reduction in neurotransmission between the various neurons. For example, a composition may contain gamma aminobutyric acid (GABA) type A receptor modulators that enhance the inhibitory effects mediated by the GABAA receptor. The GABAA receptor inhibits neuronal activity by effectively shunting current flow across the cell membrane. GABAA receptor modulators may enhance the inhibitory effects of the GABAA receptor and reduce electrical or chemical signal transmission from the neurons. Examples of GABAA receptor modulators include benzodiazepines, such as diazepam, oxaxepam, lorazepam, prazepam, alprazolam, halazeapam, chordiazepoxide, and chlorazepate. Compositions may also contain glutamate receptor modulators that decrease the excitatory effects mediated by glutamate receptors. Examples of glutamate receptor modulators include agents that inhibit current flux through AMPA, NMDA, and/or kainate types of glutamate receptors. The compositions may also include agents that modulate dopamine receptors, such as antipsychotics, norepinephrine receptors, and/or serotonin receptors. Compositions of embodiments of the invention can also include agents that affect ion flux through voltage gated calcium channels, potassium channels, and/or sodium channels.

In certain other embodiments, a combination of Botulinum toxin and endopeptidase is used in the treatment of autonomic nervous system disorders such as irritable bowel syndrome. In certain embodiments the combination of Botulinum toxin and endopeptidase is administered to the sympathetic ganglia (e.g. superior mesenteric ganglia) of a patient suffering from a gastric disorder (e.g. irritable bowel syndrome). The combination of Botulinum toxins and endopeptidases allows for dose reduction of active agents (with associated reduction in side effects) as well as possible synergistic effects. Non-paralytic effects, and also possible prophylactic effects especially when used early in the condition can provide further benefits. One difference between re-targeted endopeptidases such as TVEMPs and native clostridial toxins is that because the TVEMPs do not target motor neurons, the lethality associated with over-dosing a mammal with a TVEMP is greatly minimized, if not avoided altogether. For example, opioid TVEMPs can be administered at 10,000 times the therapeutically effective dose before evidence of lethality is observed, and this lethality thought to be due to the passive diffusion of the molecule and not via the intoxication process. Thus, for all practical purposes TVEMPs are non-lethal molecules. Brainstem over-activity can be attenuated by sensory modulation via the use of TVEMPs. Whereas Botolunim toxins have predominantly motor effects, TVEMPs may control similar conditions by decreasing sensory afferents. Non-paralytic effects and also possible prophylactic effects, especially when used early in the condition, are additional benefits. By using a combination therapy of Botulinum toxin with TVEMPs, a lower dose of the toxin can be used to treat the disorder. This can result in a decrease in muscle weakness generated in the compensatory muscles relative to current treatment paradigms. The assessment for injection would require a careful examination for agonists and compensatory painful muscles. The molar ratio of Botulinum toxin to TVEMP in the combination treatment of certain embodiments may be a 1:1 ratio; a 1:2 ratio; a 1:5 ratio; a 1:10 ratio; a 1:20 ratio; a 1:50 ratio; a 1:100 ratio; 1:200 ratio; a 1:500 ratio; a 1:1000 ratio; 1:2,000 ratio; a 1:5,000 ratio; or a 1:10,000 ratio. Endopeptidases, such as TVEMPs which target sensory nerve endings, can decrease the effects of the vagus nerve as well as its brainstem and cortical connections. Modulation of the vagus nerve can benefit patients suffering from seizure disorders, depression, chronic pain, hiccups, chronic cough, nausea and vomiting, as well as other disorders of gastro-intestinal and genito-urinary motility origin. Vagus overactivity can be attenuated by sensory modulation such as that achieved by decreasing sensory afferents, such as through the use of TVEMPs. A TVEMP can bind to a target cell, translocate into the cytoplasm, and block the release of acetylcholine (ACh) at the pre-synaptic neuromuscular junction. Non-paralytic effects, and also possible prophylactic effects, are additional benefits of the TVEMP treatment. These endopeptidases can be delivered to the target sites by transdermal or injectable methods.

Implants useful in certain embodiments of the invention may be prepared by mixing a desired amount of a stabilized Botulinum toxin (such as non-reconstituted BOTOX®) into a solution of a suitable polymer dissolved in methylene chloride. The solution can be prepared at room temperature. The solution can then be transferred to a Petri dish and the methylene chloride evaporated in a vacuum desiccator. Depending upon the implant size desired and hence the amount of incorporated neurotoxin, a suitable amount of the dried neurotoxin-incorporating implant is compressed at about 8000 p.s.i. for 5 seconds or at 3000 p.s.i. for 17 seconds in a mold to form implant discs encapsulating the neurotoxin. See e.g. Fung L. K. et al., Pharmacokinetics of Interstitial Delivery of Carmustine 4-Hydroperoxycyclophosphamide and Paclitaxel From a Biodegradable Polymer Implant in the Monkey Brain, Cancer Research 58;672-684:1998.

The dose of a Botulinum toxin administered according to the present invention can vary widely according to various patient variables including size, weight, age, disease severity, responsiveness to therapy, and solubility and diffusion characteristics of the Botulinum toxin chosen. Methods for determining the appropriate route of administration and dosage are generally determined on a case by case basis by the attending physician. Such determinations are routine to one of ordinary skill in the art. In certain embodiments the dose of Botulinum toxin can be administered in an amount of between about 1 unit and about 3,000 U; and the effect of the administration can persist for between about 1 month and about 5 years.

In some embodiments, a physician may have to alter dosage in each case in accordance with the assessment of the severity of the condition, as typically done when treating patients with a condition/disorder. Further, in some embodiments, the treatment may have to be repeated at least one additional time, in some cases several times, depending on the severity of the condition and the patient's overall health. If, for example, a patient is not deemed physically suitable for a full administration of botulinum toxin, or if a full administration is not desired for any reason, smaller doses on multiple occasions may prove to be efficacious. In certain embodiments, the treatment is the sole treatment administered to the patient.

Of course, an ordinarily skilled medical provider can determine the appropriate dose and frequency of administration(s) to achieve an optimum clinical result. That is, one of ordinary skill in medicine would be able to administer the appropriate amount of the toxin, for example Botulinum toxin type A, at the appropriate time(s) to effectively treat the disorder. The dose of the neurotoxin to be administered depends upon a variety of factors, including the severity of the disorder. The dose of the toxins employed in accordance with this invention may be equivalent to the dose of BOTOX® used in accordance with the present invention described herein. In various methods of the present invention, from about 0.01 U/kg (U of botulinum toxin per kilogram of patient weight) to about 15 U/kg, of a BOTOX® e.g. botulinum toxin type A, can be administered. In some embodiments, about 0.1 U/kg to about 20 U/kg of BOTOX® may be administered. Use of from about 0.1 U/kg to about 30 U/kg of a BOTOX®, is within the scope of a method practiced according to the present disclosed invention. In one embodiment, about 0.1 U/kg to about 150 U/kg botulinum toxin, for example type A, may be administered.

Certain embodiments of the present invention provide methods for treating irritable bowel syndrome that include the administration of a Botulinum toxin to the superior mesenteric ganglia and/or to the region thereof. Administration of a Botulinum toxin to the superior mesenteric ganglia and/or region thereof can result in increased gut motility, which could lead to the reversal of constipation and abdominal pain.

In various administration methods of the present invention, needles of various sizes can be utilized. In certain embodiments, the needle used is at least about 1 inch long. In certain preferred embodiments, the needle used is from about 1 inch to about 2 inches long. Further, in certain embodiments the needle used is a 30, 27, 25 or 22 gauge needle.

The depth of insertion of the needle is dependant upon needle length and patient anatomy. In some embodiments the needle is inserted to its full length.

In certain embodiments, the needle is a hollow hypodermic needle that is slightly curved at the end. In preferred embodiments, the needle is a Tuohy needle.

In preferred embodiments, the patient is in a prone position during treatment.

In certain embodiments the needle is placed at between about 4 cm to about 10 cm lateral to the L3 spinous process. In preferred embodiments, the needle is placed at about 7 cm lateral to the L3 spinous process.

In certain embodiments the needle is then advanced from about 1 cm to about 3 cm anterior to the L3 vertebral body under fluoroscopy, direct vision, direct surgical vision, or endoscopic delivery. In preferred embodiments, the needle is then advanced about 2 cm anterior to the L3 vertebral body with fluoroscopic guidance.

In certain embodiments, after needle insertion, from about 50 units to about 150 units of a Botulinum toxin is injected in the region of the superior mesenteric ganglion. In preferred embodiments about 100 units of Botulinum toxin type A is injected in the region of the superior mesenteric ganglion.

Upon injection of the desired volume of Botulinum toxin, the needle is withdrawn.

While injection of a Botulinum toxin into the region of the superior mesenteric ganglion is described herein, administration of a Botulinum toxin to the region of the inferior mesenteric ganglion, as described herein, is also contemplated.

In certain embodiments, a syringe containing a concentrated solution of Botulinum toxin type A, and a 1.5 inch, 27 gauge needle, is used. A concentrated solution of Botulinum toxin is preferably used, such as, for example and in the case of utilizing BOTOX® (Botulinum toxin Type A), 2 cc of normal unpreserved saline per 100 unit vial of BOTOX®. In certain embodiments, from about 1 cc to about 4 cc dilutions per 100 units of BOTOX® are used.

Compositions and methods according to the invention disclosed herein have many advantages, including that a botulinum toxin can be used to provide therapeutically effective treatment of an autonomic nervous system disorder such as irritable bowel syndrome. All references, articles, publications and patents and patent applications cited herein are incorporated by reference in their entireties. Although the present invention has been described in detail with regard to certain preferred methods, other embodiments, versions, and modifications within the scope of the present invention are possible.

Accordingly, the spirit and scope of the following claims should not be limited to the descriptions of the preferred embodiments set forth above.

### EXAMPLE 1

### The Use of Botulinum Toxin Type A in the Treatment of Irritable Bowel Syndrome

A patient suffering from irritable bowel syndrome is placed in a prone position, and a Tuohy needle is placed at 7 cm lateral to the L3 spinous process. The needle is advanced 2 cm anterior to the L3 vertebral body with fluoroscopic guidance. Botulinum toxin type A 100 units, dissolved in 2 cc of normal saline is injected around the superior mesenteric ganglion.

What is claimed is:

1. A method for treating a gastric disorder, comprising:
administering a botulinum toxin to a sympathetic ganglion of a patient suffering from the gastric disorder, thereby treating the patient.

2. The method of claim 1, wherein the botulinum toxin is selected from the group consisting of botulinum toxin types A, B, C, D, E, F and G.

3. The method of claim 1, wherein the botulinum toxin is a botulinum toxin type A.

4. The method of claim 1, wherein the botulinum toxin is a botulinum toxin complex.

5. The method of claim 1, wherein the botulinum toxin is a pure botulinum toxin.

6. The method of claim 1, wherein the gastric disorder is irritable bowel syndrome.

7. The method of claim 1, wherein the sympathetic ganglion is a mesenteric ganglion.

8. The method of claim 7, wherein the mesenteric ganglion is a superior mesenteric ganglion.

9. The method of claim 7, wherein the mesenteric ganglion is an inferior mesenteric ganglion.

10. The method of claim 1, wherein the botulinum toxin is administered in an amount between about 1 unit and about 3,000 units.

11. The method of claim 10, wherein the botulinum toxin is administered in an amount of about 100 units.
